(19) United States
(12) Patent Application Publication
(10) Pub. No.: US 2006/0241396 A1
(43) Pub. Date: Oct. 26, 2006

(54) Multi-modal detection of surgical sponges and implements

(76) Inventors: Carl E. Fabian, Miami, FL (US); Dave Narasimhan, Flemington, NJ (US); Ernest D. Buff, Far Hills, NJ (US)

Correspondence Address: ERNEST D. BUFF;ERNEST D. BUFF AND ASSOCIATES, LLC., 231 SOMERVILLE ROAD, BEDMINSTER, NJ, 07921, US

(21) Appl. No.: 11/054,844

(22) Filed: Feb. 10, 2005

(51) Int. Cl. A61B 5/05; G08B 13/14

(52) U.S. Cl. 600/424; 340/572.1

(57) ABSTRACT

A multi-modal detection system is appointed to reliably detect metallic surgical instruments such as scalpels, hemostats and the like, and non-metallic surgical implements such as sponges or laparotomy pads and gauze pad associated with an embedded marker. A first system detects metallic surgical instruments and a second independent system detects non-metallic surgical implements. The systems operate sequentially. The metallic surgical implements are removed immediately after detection and prior to scanning by the second system. Shielding of markers embedded in non-metallic surgical implements is thereby prevented. The marker may be a mechanically resonant target or RFID target. The first and second systems may be attached to be a rollaway cart and can comprise hand held antenna.

### BACKGROUND OF THE INVENTION

1. Field of the Invention

The present invention relates to a system for detecting metallic and non-metallic surgical implements; and more particularly, to a multi-modal system having a first metal detecting modality for detection of metallic surgical implements and at least a second detecting modality, operative sequentially with said first detecting modality, for detection of non-metallic surgical implements such as surgical sponges and the like.

2. Description of the Prior Art

Many patents disclose methods for detection of surgical implements following surgery prior to wound closure. Such detection methods incorporate x-ray opaque markers within surgical implements and effect detection using postoperative x-ray of the patient or of discarded sponges. Also disclosed as being suitable for detection of surgical implements are methods involving use of resonant tags made from magnetomechanical elements, capacitors, LRC oscillatory circuits and smart markers.

U.S. Pat. No. 3,097,649 to Gray discloses a method and application for detecting a surgical sponge. The sponge carries a radioactive material such as uranium oxide sewn therein. Radiation emitted from the sponge is detected by a Geiger counter prior to surgical wound closure. No disclosure is contained therein concerning a method for detection of retained metallic implements.

U.S. Pat. No. 3,508,551 to Walters et al. discloses dressings and production thereof. An x-ray detectable opaque filament is incorporated within the dressing. Use of this device, the patient must be transported from the operating room to an x-ray room. The process is cumbersome and exposes the patient to unnecessary radiation. Detection of a retained dressing tends to be limited due to the small diameter of the x-ray opaque filament. The dressing can be overlooked when orientation of the filament is directly in-line with a bone.

U.S. Pat. No. 3,587,583 to Greenberg discloses a surgical sponge with magnetizable means. The sponge has a flexible thread with magnetizable particles. A plurality of magnetizable barium ferrite particles is embedded in a plastic material such as nylon, forming a flexible magnetizable thread. The surgical instruments used may also be provided with a small amount of magnetizable material. A surgical cavity is probed using a magnetic detection means such as a magnetodiode. The Greenberg disclosure does not state how the surgical instruments could be made to have magnetizable material. The size of threads is too small to be detected unless the probe is also inserted into the surgical cavity, which procedure would likely present issues involving sterility and tissue damage.

U.S. Pat. No. 3,686,564 to Mallick, Jr. et al. discloses a multiple frequency magnetic field technique for differentiating between classes of metal objects. Low and high frequency oscillators are mixed using multiple frequency excitation. The magnetic field generated is examined by observing the voltage current vectorial relationship. When metal is present in the incident electromagnetic field, the vectorial relationship is changed according to the size, shape and type of a metallic object. The system is primarily designed to detect large metallic objects, such as guns in an airport, and is a walk through arrangement. No disclosure is set forth concerning detection of small metallic objects such as a surgical implement in a surgical incision.

U.S. Pat. No. 3,698,393 to Stone (hereinafter the '393 patent) discloses a surgical pad. A radiation opaque plastic ring is attached to the pad. After a surgical procedure is complete, the patient is radiographed to determine whether a sponge having an attached plastic ring has been retained within the surgical cavity. The system requires that the patient be transported to an X-ray facility; it exposes the patient to unnecessary radiation.

U.S. Pat. No. 3,834,390 to Hirsch (hereinafter the '390 patent) discloses a neurological sponge. The sponge has a double layer comprising a highly absorbent inner layer wrapped by a porous outer layer. An x-ray detectable BaSO4 material wrapped in plastic is placed between the two layers. The x-ray detectable material absorbs x-rays, indicating the presence of the sponge within a surgical cavity. Prior to closure of the surgical incision, the patient must be transported to an x-ray facility, at which location the patient is exposed to unnecessary x-ray radiation.

U.S. Pat. No. 3,964,041 to Hinds discloses an article detection system and method. Articles such as container ends are sensed and detected to provide count and/or control outputs representative of the number of such articles detected. A frequency sensitive detector circuit generates a fixed frequency signal, which is utilized to modulate the output from a signal generating transducer or signal source. The modulated output from the source impinges on an article being sensed, which reflects or interrupts this signal. The reflection or interruption is sensed by a suitable transducer or sensor. A feedback signal generated by the transducer or sensor is fed back to the frequency sensitive detector that generated the original fixed frequency signal. The detected signal is the same as the frequency of the original modulating signal. One of these output signals is indicative of the detection of an article, and is applied to suitable counting and/or control circuitry that provides the desired count and/or control outputs. A feedback signal indicates the presence of an article and provides an accurate count of articles such as can ends. The article detection system and method disclosed by the Hinds disclosure does not detect metallic objects or non-metallic sponges inadvertently retained within a surgical incision.

U.S. Pat. Nos. 4,114,601 and 4,193,405 to Abels disclose a medical and surgical implement detection system. Surgical implements, surgical instruments, surgical sponges, surgical implantable devices and indwelling therapeutic devices and materials are detected within the human body or other area of interest by incorporating or adding a radio frequency transponder. This is a microwave system that mixes two fundamental microwaves having 4.5-5 GHZ frequencies, and relies on a non-linear transponder to produce higher order product frequencies. The transponder may be a thin film of a ferrite material exhibiting gyro-magnetic resonance at selected frequencies. Alternately, the transponder may be a solid-state device containing diodes and field effect transistors. This non-linear transponder signal is received by a receiving antenna and is filtered to remove all fundamental microwave frequencies. Each of the higher order microwave frequencies generated by the transponder is easily absorbed by the human body. Consequently, most of the signal is lost before any non-linear transponder can be detected. In addition, the gyro-magnetic effect produces only a weak signal.

U.S. Pat. No. 4,658,818 to Miller, Jr., et al. discloses an apparatus for tagging and detecting surgical implements. A miniature battery-powered oscillator is attached to each surgical implement and activated prior to its initial use. The output of each oscillator is in the form of a low powered pulse of 1-10 MHZ frequency and is coupled to the body's fluids and tissue. After the surgery is completed, but prior to suturing, a detection system is used to sense for any pulses generated by the oscillator within the body. The surgical implement detection system disclosed by the '818 patent is not passive. It requires a miniature battery, which remains in the “on” condition from the beginning of the operation. If a sponge is left behind, the microwave radiation is detected. However, this sponge detection system is not passive. It requires a miniature battery, which remains constantly in the “on” condition from the beginning of the operation. When the operation is complete, the battery might have discharged, in which case the sponge would not be detected. In addition, the system disclosed by the '818 patent does not detect metallic objects.

U.S. Pat. No. 5,057,095 to Fabian discloses a surgical implement detector utilizing a resonant marker for use in human or animal tissue. The marker is set into resonance by the interrogating field and the resonance frequency signal emitted by the marker is detected by a separate detection circuit adjacent to the interrogating circuit. The marker resonates due to magnetostriction properties of an amorphous ribbon or piezoelectric device or a tuned LRC circuit. The marker is a single function device, and the system only detects a marker that has been incorporated in a surgical implement. It does not detect metallic objects, without a marker. Even if a marker is associated with a metallic object, the metal present in such object may shield the marker from the interrogating electromagnetic field.

U.S. Pat. No. 5,099,845 to Besz et al. discloses a medical instrument location means. A powered radiating element is attached to a device appointed for insertion into the body. The location of the radiating element within the body is assessed by moving a hand held receiving unit on the external surface of the body to obtain a maximum radiation value, thereby pointing the receiving sensor directly above the radiating element. Next, the intensity of the radiation energy is assessed to determine how deep the radiating element is located from the surface of the body. The radiating element requires power to operate and therefore does not detect unpowered metallic objects or sponges even if they contain a passive tag.

U.S. Pat. No. 5,541,604 to Meier discloses transponders, Interrogators, systems and methods for elimination of interrogator synchronization requirement. A Radio Frequency Identification (RFID) system has an interrogator and a transponder. The interrogator has a first tuned circuit of a powering frequency for sending a powering burst to a transponder. A filter/demodulator receives a wireless, modulated RF response from the transponder. The interrogator also has a second tuned circuit in electrical communication with a modulator. The second tuned circuit has a selected bandwidth about a communication frequency. The selected bandwidth does not substantially overlap the powering frequency; but encompasses the bandwidth of the modulated carrier of the RF response. The carrier is modulated using pulse width modulation (PWM), pulse position modulation (PPM), frequency-shift keying modulation (FSK), or other modulation method. The interrogator also has a controller in electrical communication with the filter/demodulator and the tuned circuits for enabling the first tuned circuit to send the powering burst during a first time period and of enabling the modulator in electrical communication with the second tuned circuit to receive the RF response during a second time period. The transponder has a tuned circuit, a tuning circuit in electrical communication with the tuned circuit for modifying the frequency characteristics of the tuned circuit such that it is can be tuned during the powering burst to the powering frequency, and be tuned during the RF response to the communication frequency. The transponder also includes a demodulator in electrical communication with the tuned circuit for receiving the RF interrogation therefrom and for demodulating data from the RF interrogation. This current generation RFID device sends a preset code to the interrogator and is powered entirely by the power burst signal provided during the first time period. It is capable of transmitting the code at a high rate to the interrogator.

U.S. Pat. Nos. 5,650,596 and 5,923,001 to Morris, et al. disclose an automatic surgical sponge counter and blood loss determination system. Each sponge carries an RF tag which is read by a sensor located in proximity with the opening of a soiled sponge-receiving container provided with a disposal bag. The disposal bag is weighed and its dry weight compared based on the ID of the sponge tag. The weight of blood and other body fluids is determined by subtraction. A display is used to provide information about sponges in the container, and the weight of blood and body fluids dispensed within the container. This system does not detect sponges retained within a patient during an operation; it only counts surgical implements when they are disposed within the container.

U.S. Pat. No. 5,944,023 to Johnson et al. discloses systems and methods for determining the location of an implanted device including a magnet. The tip of the body inserted implanted device includes a generating mechanism which may be a permanent magnet or a permanent direct current magnet with a self-induced magnetic field. The location of the magnet is detected outside the patient by a mat, which incorporates a multitude of magnetic field sensors. The magnet positional information is displayed on a video screen. This system disclosed by Johnson does not locate surgical instruments or sponges within a surgical cavity.

U.S. Pat. Nos. 6,009,878 and 6,305,381 to Weijand et al. disclose a system for locating an implantable medical device. This system has an implanted coil, which transmits electromagnetic radiation and is picked up by an electromagnetic energy receiving device with three symmetrically oriented coils external to the patient. When the energies received by these three coils are equal, the receiving device is directly above the implanted coil, and the drug reservoir in the implant may be filled. This system does not detect medical instruments or sponges accidentally retained by the surgical wound of a patient during an operation.

U.S. Pat. No. 6,057,756 to Engellenner discloses electronic locating systems. Coded tags are interrogated at various locations in the intended path of a transportation vehicle. The presence of a vehicle in a specific location is determined and relayed to a central controller. The '756 patent discloses a system for managing and tracking a transportation process. No disclosure is contained within the '756 patent concerning detection of metallic objects or sponges accidentally left behind in a surgical incision after completion of surgery.

U.S. Pat. No. 6,076,007 to England et al. discloses a portable unit for detecting the presence of surgical devices, and their location. High permeability, low coercivity, wire or strip tag is implanted with a surgical device. The tag is interrogated by a search coil energized by a high frequency AC field with a DC or low frequency bias filed. Phase information is used to detect the directionality of the tag location. The detection system is based on flying null technology. It is a single functionality detection system, and does not detect metallic objects that are not incorporated with a tag. Metallic objects adjacent to the tag may distort phase information providing an unreliable indication.

U.S. Pat. No. 6,615,155 to Gilboa discloses object tracking using a single sensor or a pair of sensors. The three dimensional movement of a moving object is tracked by measuring one or more vector fields assisted by theoretical computations. The system does not track or detect stationary objects such as a sponge or metallic object accidentally included in a surgical incision.

U.S. Pat. No. 6,026,818 to Blair, et al discloses a tag and detection device. An inexpensive tag has the form of a ferrite bead with a coil that resonates at a particular frequency, or a flexible thread composed of a single loop wire and capacitor element. The detection device locates the tag by pulsed emission of a wide band transmission signal. The tag resonates with a radiated signal, in response to the wide band transmission, at its own single non-predetermined frequency, within the wide band range. This system does not detect untagged, metallic surgical implements.

U.S. Pat. No. 6,424,262 and US Patent Application No. 20040201479 to Garber, et al. disclose applications for radio frequency identification systems. An RFID target cooperates with a magnetic security element and a bar code reader to check out and manage library materials such as reference books, periodicals, and magnetic and optical media. No disclosure is contained with the '262 patent and '479 patent Application concerning detection of sponges or surgical pads in a surgical wound.

US Patent Application No. 20030066537 to Fabian, et al. discloses surgical implement detection system. Surgical implements used during an operating procedure are detected in human tissue. Markers attached to the surgical implements change their impedance at a preselected frequency in the presence of an electromagnetic field. The system uses a magnetomechanical element which vibrates at a preselected frequency when excited, and this preselected frequency is detected, indicating the presence of a surgical implement to which the magnetomechanical marker element is attached. Such a system does not detect untagged metallic surgical implements.

US Patent Application No. 2003/0176785 to Buckman et al. discloses a method and apparatus for emergency patient tracking. This tracking system attaches a coding device to a patient and is tracked. In fact, the coded device utilized is associated with each patient in such a way that the device cannot be removed or disassociated from the patient without a concerted effort. Such a system does not detect accidentally included sponges or metallic objects in a surgical incision.

PCT Patent Application No. WO 98/30166 and European Patent Specification 1 232 730 A1 to Fabian et al. disclose a surgical implement detector utilizing a smart marker. The marker is coded and the code is transmitted through an antenna to a central microprocessor, which verifies the code. Each marker has to be individually coded and inserted into a sponge surgical pad, etc. The system does not detect untagged, metallic objects left behind within a surgical incision.

PCT Patent Application No. WO 03/032009 to Fabian et al. discloses a surgical implement detection system. A marker attached to the surgical implement changes its impedance at a preselected frequency in the presence of an electromagnetic interrogating field. The interrogating electromagnetic field has a preselected frequency, preferably modulated as a series of pulses and the marker, a magnetomechanical element, attached to the surgical implement resonates at a preselected frequency in response to the field. The detector detects a ring-down signal of the marker between the pulses. This system does not detect metallic objects that do not have a marker attached. Besides, a metallic surgical implement may shield a marker, producing a weak perselected frequency signal.

PCT Patent Application No. 03/048810 and US Patent Application No. 20030105394 to Fabian et al. disclose a portable surgical implement detector. The portable detector interrogates a marker that is attached with a surgical implement which signals a preselected frequency. This system does not detect metallic objects that do not have a marker attached. Besides, a metallic surgical implement may shield a marker, producing a weak preselected frequency signal.

US Patent Application No. 20030192722 to Ballard discloses a system and method of tracking surgical sponges. The sponges have a radiopaque object embedded and is visible when the sponge container is x-rayed. Each of the sponges that has been brought into the operating room is x-ray identified. A missing sponge is detected by this accounting process. However, the process does not actively detect whether a sponge is accidentally left behind in a surgical wound. The patient is not x-rayed to determine whether the missing sponge is within the patient.

US Patent Application No. 20040129279 to Fabian, et al. discloses a miniature magnetomechanical tag for detecting surgical sponges and implements. This tag is a magnetomechanical device, and is excited by the interrogating magnetic field. The interrogating field is switched off and the ring down of the resonant target is detected. This system does not provide digital means for identifying a sponge or surgical pad.

US Patent Application No. 20040250819 to Blair, et al discloses an apparatus and method for detecting objects using tags and wideband detection devices. An apparatus and method for the detection of objects in the work area such as surgical sites, including a detection tag affixed to objects such as used during surgery, is disclosed. The apparatus and method feature interrogates with a transmitter emitting a pulsed, wideband signal. This signal prompts the tag element to provide a return signal, which is received and analyzed. The device features an antenna portion containing a single or a plural ring-shaped antenna. Also, the pulsed wideband interrogation signal may be pulsed-width modulated or voltage-modulated. The pulsed signals trigger a continuing response signal from the tag in its response frequency range, which increases in intensity to the point where it becomes differentiable from background noise and is detected within the wideband range by the signal detector as an indication of the presence of the tag. The tag is excited by a wide band pulsed interrogation signal, which builds up the output of the tag and can be detected over ambient electronic noise. The tag signal has a predetermined frequency. It is a sinusoidal wave, and does not carry digital information identifying the sponges and surgical pads used.

US Patent Application No. 2005/0003757 to Anderson discloses an electromagnetic tracking system and method using a single-coil transmitter. The system includes a single coil transmitter emitting a signal, a receiver receives a signal from the single coil transmitter. Electronics process the signal received by the receiver. The electronics determine a position of the single coil transmitter. The transmitter may be a wireless or wired transmitter. The receiver may be a printed circuit board. The electronics may determine position, orientation, and/or gain of the transmitter. The single coil transmitter is a powered device and may be wired or wireless. It is not a passive device that can be incorporated in a sponge or surgical pad due to the requirement for a reliable power source.

PCT Patent Application No. WO 98/30166 to Fabian et al. discloses a surgical implement detector utilizing a smart marker. The surgical implement is appointed for disposition within human or animal tissue. It is caused to become electronically identifiable by affixing thereto a smart marker, which is an unpowered integrated circuit with an EEPROM memory that carries a code. When smart marker is sufficiently close to the reader antenna, a voltage is generated within the marker antenna that charges the capacitor and powers the integrated circuit. A switch is opened and closed to transmit the stored code in the EEPROM memory, providing identification and recognition of a smart target attached to a surgical sponge. The marker antenna operates at a frequency of near 125 KHz. Frequency of information transfer to the reader is very slow due to the switching on and off action. In addition, the smart marker is not encapsulated and is subject to damage by blood and other saline fluids.

There remains a need in the art for a highly reliable surgical implement detection marker that detects both metallic surgical instruments and non-metallic surgical implements including sponges, laparotomy pad and gauze, so that none of these surgical implements are left behind in a surgical incision at the end of the operation prior to surgical wound closure. The procedure for detection of accidentally included surgical implements must be exceedingly reliable. Apparatus employed to practice this procedure and be easy to operate so that the patients can be routinely examined without complex set-up and teardown steps, enabling risk of infection and rejection reactions to be minimized.

### SUMMARY OF THE INVENTION

The present invention provides a multi-modal detection system for identifying the presence of surgical sponges or laparotomy pads and implements in a surgical wound. A plurality of discrete sensing systems are provided. A first system is tailored to detect ferrous and metallic objects, and a second system is tailored to detect non-metallic objects such as sponges and laparotomy pads equipped with a marker tag in the surgical wound. Each of the systems may be powered by a battery that is, preferably, a rechargeable battery, or AC power.

The first metal detecting system comprises conventional electronic circuitry adapted for the detection of metal objects. The first system includes a first detection means, which comprises first field generating means in a first antenna for generating electromagnetic radiation. The electromagnetic radiation couples with any metallic instruments within the surgical wound and the overall inductance of the first antenna is accordingly increased. The first system analyzes this change in inductance to detect the presence of metallic instruments within a surgical wound.

Preferably, the metal-detecting circuitry includes a pulse interrogation system, wherein a search coil is energized by a current pulse. The magnetic field emanating from the coil induces eddy currents in a nearby conductive object. When the interrogating magnetic field is pulsed, those eddy currents in the metallic object, in turn, produce a decaying magnetic field, which may be detected by voltage induced in a detection coil. The same coil may be used both as the search coil and the detection coil. Other known metal-detecting circuit configurations may also be used. It is preferred that the metal-detecting circuitry be able to detect both ferromagnetic objects (e.g., those containing iron, steel, nickel, or cobalt) and non-magnetic metals such as copper, aluminum, many stainless steels, titanium, superalloys, and the like.

The second system detects markers or tags incorporated within non-metallic objects such as sponges and surgical pads in a surgical wound. A marker is integrally incorporated in a glass or polymeric liquid tight package that is resistant to washing and laundering as well as sterilization procedures. A number of marker-detecting modalities may also be employed, including mechanically resonant markers; “smart” RF markers; or commercially available RFID targets. The second system includes a second detection means, which comprises second field generating means in a second antenna for generating electromagnetic radiation. The electromagnetic radiation couples with markers embedded in or attached to sponges or laparotomy pads, gauze pad within the surgical wound and the second antenna receives the response from the marker. The second system analyzes this response to detect the presence of a marker embedded in or attached to non-metallic surgical implements within a surgical wound.

In a first aspect of the second system, the marker exhibits mechanical resonance at a resonant frequency in response to the incidence thereon of an alternating electromagnetic interrogating field, whereby the marker is provided with a signal-identifying characteristic. The resonance is preferably detected by providing the interrogating field in the form of a pulse and sensing the ring-down decay in amplitude of the electromagnetic signal transmitted by the resonating marker. The marker may be comprised of magnetostrictive amorphous strip, a piezoelectric crystal circuit or a tuned LCR circuit, which has a characteristic resonance frequency. The first system operates at a frequency of 50 KHz to 150 KHz and preferably at a frequency of 100 KHz to 125 KHz.

In a second aspect of the second system, the marker has an antenna and a memory for storing a predetermined code. The marker is powered by a voltage induced in the antenna by the electromagnetic interrogating field. It is operative in the presence of the interrogating field to transmit the predetermined code as a change in the impedance of the antenna. One embodiment of the marker detection system related to the second aspect is disclosed by EP 0 967 927 B1 to Fabian and Anderson. In an alternate embodiment, the marker may be a commercial RFID tag which transmits the code as a modification of the carrier signal frequency in the range of 13.56 MHz to 2.456 GHz by pulse width modulation (PWM), pulse position modulation (PPM), frequency-shift keying modulation (FSK).

Generally stated, the invention involves the use of two discrete systems to identify surgical implements. The first system detects metallic objects, which do not generally carry a marker or a tag. The second system detects non-metallic objects such as sponge or laparotomy pad, gauze pad within which a marker or tag is embedded. This marker is interrogated by an antenna of the second system, which generally uses radio frequency electromagnetic waves. Metallic objects, depending upon their size, may shield a radio frequency marker that is in close proximity with the metallic object. In the preferred mode of operation of the multi-modal detection system, the first metallic detector is used to detect and remove the metallic object. Thereafter, the second system is used to detect the presence of any non-metallic objects with embedded markers. Use of the system therefore eliminates any possibility that surgical implements, metallic or non-metallic, have been left behind within a surgical cavity.

The first and second detector may be conveniently mounted on a rollaway cart with antennas that are fixed to the cart or attached to a hand held sensor coil that is scanned, or moved, over the surgical cavity. The sensor coil of the first system is used first to detect and remove metallic objects. Following removal of metallic objects, the sensor coil of the second system is actuated to detect non-metallic objects associated with the encapsulated marker.

Metal detectors use different physical principles to detect a metallic object. Typically, an AC circuit with a coil acts as a transmitted antenna. When a metallic object is brought in close proximity, eddy currents are induced in the metallic object, thereby increasing the inductance of the search coil. Such increase in induction is detected as a change in the voltage-current characteristics. The circuit may look for changes in the voltage-current relationships. It detects the metallic object only when the sensor coil is swept across with a very high level of sensitivity. If the sensor coil is maintained stationary, it will no longer observe a change of inductance in the coil. Since the metal detector of the first system using this type of sensor coil circuit generally requires some movement of the antenna with respect to the surgical cavity, the antenna of the first system attached to a cart may be energized as the cart is moved into position next to the patient. Such movement is sufficient to establish whether a metallic instrument is left behind within a surgical cavity. When a hand held version of the sensor coil is used in the first system, the movement of the sensor coil of the first system over the surgical cavity detects the accidentally included metallic surgical instrument. Preferably, the first system configuration uses a pulsed interrogating field. The same antenna or sensor coil looks for a decay in the eddy current of the metallic object. Advantageously, the preferred system does not require movement of the sensor coil.

The second system, which detects non-metallic surgical implements having an embedded marker, does not require antenna movement. An antenna fixed to the rollaway cart is generally adequate. However, a hand held sensor coil may also be provided in the second system for convenience and ease of use.

### BRIEF DESCRIPTION OF THE DRAWING

The invention will be more fully understood and further advantages will become apparent when reference is had to the following detailed description of the preferred embodiments of the invention and the accompanying drawing, in which:

FIG. 1 is a schematic diagram showing a multi-modal detection system;

FIG. 2A is a schematic diagram showing an RFID target;

FIG. 2B is a schematic diagram showing a magnetomechanical resonance target;

FIG. 3A is a diagrammatic representation of a sponge surgical implement provided with a magnetomechanical resonance marker tag and an RFID tag; and

FIG. 3B is a diagrammatic representation of a gauze pad surgical implement provided with a magnetomechanical resonance marker tag and an RFID tag.

### DETAILED DESCRIPTION OF THE INVENTION

The invention provides a multi-modal detection system for identifying the presence of surgical sponges and implements. The system has a plurality of discrete sensing systems, a first system being tailored to detect ferrous and other metallic objects, and a second system tailored to detect non-metallic objects such as sponges or laparotomy pad and gauze pads equipped with a marker tag. Both systems are powered by a battery, which is preferably a rechargeable battery, or AC power.

The first metal detecting system comprises conventional electronic circuitry adapted for the detection of metal objects in the surgical wound. The first system includes a first detection means, which first field generating means in a first antenna for generating electromagnetic radiation. The electromagnetic radiation couples with any metallic instruments within the surgical wound and the overall inductance of the first antenna is accordingly increased. The first system analyzes this change in inductance to detect the presence of metallic instruments within a surgical wound. The electronic circuit of the metallic object detector may detect the change in the inductance of the sensor coil; the change of phase of voltage impressed, or current passing through the search coil, or rate of change of current or voltage as a sensor coil is swept over a metallic object. Preferably, the metal-detecting circuitry includes a pulse interrogation system, wherein a search coil is energized by a current pulse. After the pulse is interrupted, the decaying magnetic field emanating from the coil induces eddy currents in a nearby conductive object. Those currents, in turn, produce a decaying magnetic field, which may be detected by voltage induced in a detection coil. In some embodiments, the same coil may be used both as the search coil and the detection coil. Other known metal-detecting circuit types may also be used. It is preferred that the metal-detecting circuitry be able to detect both ferromagnetic objects (e.g., those containing iron, steel, nickel, or cobalt) and non-magnetic metals such as copper, aluminum, many stainless steels, titanium, superalloys, and the like.

The following examples are presented to provide a more complete understanding of the invention. The specific techniques, conditions, materials, proportions and reported data set forth to illustrate the principles and practice of the invention are exemplary and should not be construed as limiting the scope of the invention.

### Example 1

A metal detector system ‘Auto Scan Security Detector’ manufactured by White Electronics Inc. is used to detect metallic objects in a surgical incision in a cadaver. The Auto Scan detector is positioned at a distance from 0.5 inches to 7.5 inches from the cadaver body surface and antenna was scanned back and forth. This movement is essential to detect the metallic objects, since the movement results in a change in the magnetic coupling between the antenna and a metallic object resulting in a audible signal. If the metallic object is oriented perpendicular to the surgical cavity, sensitivity of detection is reduced. Since the surgical cavity is generally flat and has a limited depth, the surgical instruments tend to lie flat in the cavity and are very easily detected.

The second system detects markers or tags incorporated within non-metallic objects such as sponges or laparotomy pad and gauze pads in a surgical wound. A marker is integrally incorporated in a glass or polymeric package that is resistant to washing and laundering as well as sterilization procedures. The second system includes a second detection means, which comprises second field generating means in a second antenna for generating electromagnetic radiation. The electromagnetic radiation couples with markers embedded in or attached to sponges or laparotomy pads or gauze pad within the surgical wound and the second antenna receives the response from the marker. The second system analyzes this response to detect the presence of marker embedded in or attached to non-metallic surgical implements within a surgical wound. A number of marker-detecting modalities may also be employed, including mechanically resonant markers and “smart” RF markers such as commercially available RFID tags. The commercial RFID tags which operate without need for a battery have a capacitor circuit which is charged by an interrogating electromagnetic field carrier wave and powers an integrated chip which has a burnt-in code in a read only memory. The capacitor power is used to modulate the carrier wave to encode and broadcast the code as a pulse width modulation or pulse position modulation or frequency shift keying modulation. The modulated carrier wave is received by the interrogating antenna and is decoded to identify the code and relate it to the identity of a non-metallic object using a look-up table.

In a first aspect of the second system, the marker exhibits mechanical resonance at a resonant frequency, in response to the incidence thereon of an alternating electromagnetic interrogating field, whereby the marker is provided with a signal-identifying characteristic. The resonance is preferably detected by providing the interrogating field in the form of a pulse and sensing the ring-down decay in amplitude of the electromagnetic signal transmitted by the resonating marker. The marker may be made from magnetostrictive amorphous strip, a piezoelectric crystal circuit, or a tuned LCR circuit, which has a characteristic resonance frequency. One such magnetomechanical marker, and a surveillance system incorporating the marker, is disclosed by U.S. Pat. No. 4,510,489.

In a second aspect of the second system, the marker has an antenna and a memory for storing a predetermined code. The marker is powered by a voltage induced in the antenna by the electromagnetic interrogating field and is operative in the presence of the interrogating field to transmit the predetermined code as a change in the impedance of the antenna. One embodiment of the marker detection system related to the second aspect is disclosed by EP 0 967 927 B1 to Fabian and Anderson.

A number of manufacturers produce these radio frequency markers. Most notable of these manufacturers are Texas Instruments, Hughes Identification Devices, Destron-Fearing Corporation. Modern RFID tags also provide significant amounts of user accessible memory, sometimes in the form of read-only memory or write-once memory. The amount of memory provided can vary, and influences the size and cost of the integrated circuit portion of an RFID tag. Typically, between 128 bits and 512 bits of total memory can be provided economically. For example, an RFID tag available from Texas Instruments of Dallas, Tex., under the designation “Tag-it” provides 256 bits of user programmable memory in addition to 128 bits of memory reserved for items such as the unique tag serial number, version and manufacturing information, and the like. Similarly, an RFID tag available from Philips Semiconductors of Eindhoven, Netherlands, under the designation “I-Code” provides 384 bits of user memory along with an additional 128 bits reserved for the aforementioned types of information. Such tags operate at frequencies ranging from 125 KHz to 2.45 GHz. The lower frequency tags (about 125 KHz) are moderately resistant to shielding, but have only limited radio frequency functionality due to bandwidth constraints. In particular, systems based on these markers generally operate reliably only when a single tag is in the interrogation zone at a time. They also tend to be relatively bulky and expensive to manufacture. At higher frequencies, (typically 13.56 MHz, 915 MHz, and 2.45 GHz), the added bandwidth available has permitted the development of systems which can reliably process multiple tags in the interrogation zone in a short period of time. However, the high frequency markers are susceptible to shielding by adjacent metallic objects. In a surgical cavity large metallic objects are not present. Accordingly, the high frequency radio frequency markers are preferred, owing to their small size and reduced manufacturing cost.

Since the first system and the second system use electromagnetic radiation to detect metallic instruments and marker attached non-metallic surgical implements in a surgical wound, it is important that there is no deleterious interaction between the two systems. This is only of concern when magnetomechanical markers are used which respond with a characteristic signal frequency, which may be within the frequencies used by the first metal detecting system. The first system may be electronically programmed to disregard this resonance frequency during the process of detecting metallic objects. Alternatively, the frequencies used for metallic detection by the first system may be dissimilar from that used by the second, marker detecting system.

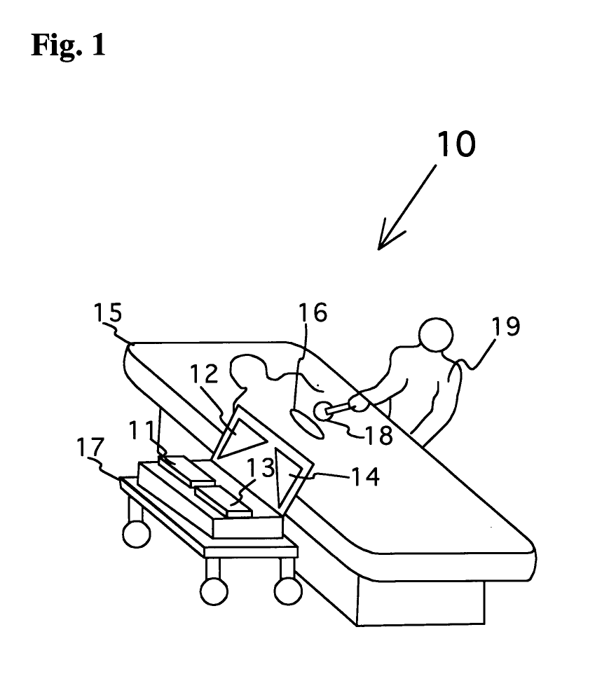

FIG. 1 is a schematic diagram showing a multi-modal detection system 10 comprising two separate detecting systems. A first system 11 and a second system 13 are placed in close proximity to an operating table 15 supporting a patient having an incision 16. The first system has an antenna 12 providing metal detection functionality, and the second system has an antenna 14 providing marker detection functionality. The multi-modal detection system may be conveniently mounted on a rolling cart 17 and brought close to the patient immediately after surgery prior to closing the incision. The first system 11 has attached or remote antenna 12 or a hand held antenna 18 manipulated by a surgeon 19. The antenna is connected to the first system 11 using a wired or remote connection, thereby forming a metal detection system. The second system 13 with antenna 14 may represent a detection system for sponges, surgical pads or gauze pad incorporated with marker tags. Thus, the first system 11 detects ferrous and non-ferrous metallic objects including surgical instruments and the like, while the second system 13 detects a marker tag attached to objects including sponges or laparotomy pads, gauze pad and the like. The multi-modal detection system 10 does not use any X-ray detection systems, and thus is highly portable into an operating room, bringing the multi-modal detection system in close proximity with the patient, when needed.

Since both the first system 11 and the second system 13 use electromagnetic waves to interrogate the surgical wound for the presence of metallic objects or included sponges prior to wound closure, there exists the potential for electromagnetic interaction between the two systems. In particular large metallic objects present in a surgical cavity may shield a marker, preventing its detection by the second system In a preferred embodiment, the two systems interrogate the surgical wound during sequential time periods so that the first, metallic object detecting system 11 detects and facilitates removal of metallic surgical instruments in the surgical cavity. Once such metallic objects are removed, the second system can reliably detect marker embedded non-metallic surgical implements that are present within the surgical cavity.

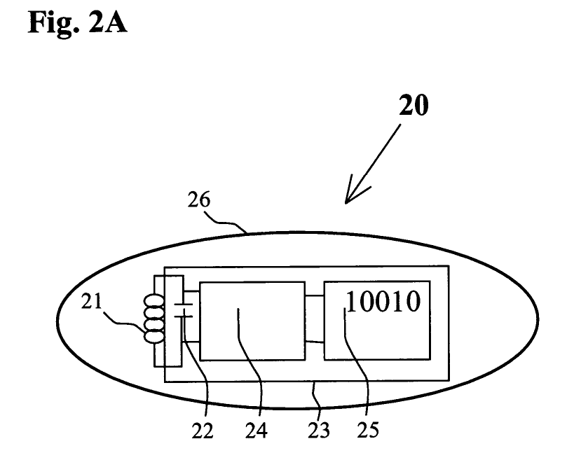

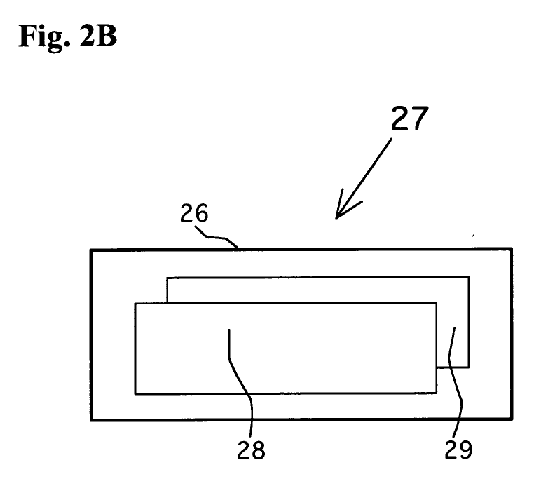

Referring to FIG. 2A, there are shown generally at 20 the details of the radio frequency marker. The marker has an antenna at 21 which receives a power pulse from a remote detector-interrogating antenna (not shown) to charge a capacitor 22. This capacitor 22 becomes the power source for the operation of the unpowered radio frequency marker, which has an integrated switch, having an integrated circuit 23 which has a reading function, a carrier frequency modulating function at 24 and a read only memory portion 25 with a burnt-in code marked here as ‘10010’. The radio frequency integrated chip, together with the antenna 21, is encapsulated in a blood, saline solution or water resistant enclosure 26.

Referring to FIG. 2B there is shown generally at 27 a magnetomechanical strip, 28 together with a biasing strip element 29 encased in a water resistant enclosure 26.

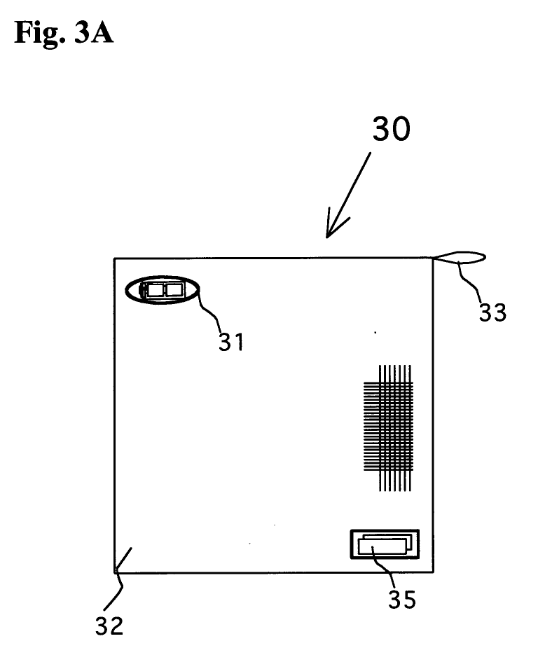

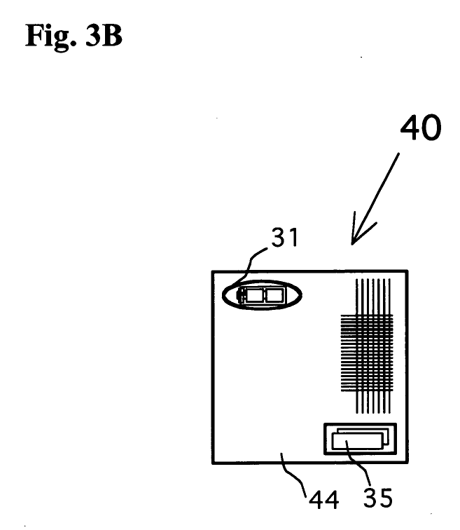

Referring to FIG. 3A, there are shown generally at 30 the details of the incorporation of the radio frequency marker in a surgical sponge or a laparotomy pad, which is typically fabricated from soft absorbent cloth, 14 or 18 inches square. The encapsulated radio frequency marker 31 is incorporated inside a sponge 32 or attached with a non-metallic thread 33. Also illustrated is a magnetomechanical resonance marker 35 incorporated in the sponge or surgical pad 32.

Referring to FIG. 3B, there are shown generally at 40 the details of the incorporation of the radio frequency marker in gauze pad, which is typically a 4 inches square. Gauze pad 44 has encapsulated radio frequency marker 31 sewn therein, as shown. Also illustrated is a magnetomechanical resonance marker 35 incorporated in the gauze pad 44.

Significant advantages are realized by practice of the present invention. The key components of a multi-modal system for detecting surgical sponges and implements, or foreign bodies accidentally included within a surgical wound, comprise, in combination, the features set forth below:
1) a battery or AC powering means;
2) a first system designed for detecting metallic objects generally having the size of commonly used surgical implements that are ferrous or non-ferrous;
3) a second system designed for detecting an included marker in non-metallic objects such as sponges or laparotomy pads, gauze pad and the like;
4) the second system marker selected from a class of markers comprising resonant markers or antenna-powered RFID markers capable of sustaining sterilization procedures;
5) each of the first and second system markers using dedicated remote antennae to interrogate a surgical wound for the presence of metallic objects and non-metallic objects containing embedded markers;
6) said first and second systems operating at different electromagnetic radiation frequencies, thereby preventing interference between metallic objects and markers present in the surgical wound;
7) the first and second systems operating sequentially so that the first system ignores the characteristic resonance frequency of the second system magnetomechanical marker when interrogating the presence of metallic objects; and
8) the sequential operation being such that metallic objects detected by scanning the surgical wound with the first system are removed from the wound prior to scanning the wound with the second system.

Having thus described the invention in rather full detail, it will be understood that such detail need not be strictly adhered to, but that additional changes and modifications may suggest themselves to one skilled in the art, all falling within the scope of the invention as defined by the subjoined claims.

What is claimed is:

1. A multi-modal system for detecting surgical sponges and implements within a surgical cavity of a patient, comprising:
a. a first system appointed to detect metallic surgical instruments;
b. a second system appointed to detect non metallic surgical implements, and comprising an embedded encapsulated marker associated with each of said non metallic surgical implements;
c. said first system having a first detection means for detecting metallic surgical instruments, said detecting means comprising first field generating means for generating an applied field containing electromagnetic radiation using a first antenna, and analyzing inductance of said first antenna to determine whether said electromagnetic radiation has been changed by the presence of said metallic surgical instrument within said applied field, and thereby detect the presence of said metallic instrument therewithin;
d. said second system having a second detecting means for detecting non-metallic surgical implements, said second detecting means comprising second field generating means for generating an applied field containing electromagnetic radiation using a second antenna, and analyzing received electromagnetic radiation from said marker using said second antenna, to detect the presence of said marker within said applied field, and thereby detect the presence of said non metallic surgical implement associated therewith;
whereby scanning a surgical wound sequentially using the first and second system prior to wound closure detects metallic surgical instruments and non-metallic surgical implements with embedded markers, thereby eliminating their accidental retention within said surgical wound.

2. The multi-modal system as recited by claim 1, wherein said first detection means is operative to monitor change in inductance of said first antenna, said change in induction indicating the presence of said metallic instrument in said applied field.

3. The multi-modal system as recited by claim 1, wherein said first detection means is operative to monitor change in phase of current in said first antenna, said change in phase of current signaling the presence of said metallic instrument in said applied field.

4. The multi-modal system as recited by claim 1, wherein said first field generating means is operative to apply a pulsed electromagnetic current to said first antenna, and said detection means is operative to monitor decay of current in said first antenna, said decay of current indicating the presence of said metallic instrument in said applied field.

5. The multi-modal system as recited by claim 1, wherein said embedded encapsulated marker is mechanically resonant, and said second detecting means is operative to detect non-metallic implements associated with said embedded encapsulated marker.

6. The multi-modal system as recited by claim 1, wherein said embedded encapsulated marker comprises a non powered RFID broadcasting code, and said second detecting means detects non-metallic implements associated with said embedded encapsulated marker.

7. The multi-modal system as recited by claim 1, wherein said embedded encapsulated marker is encapsulated by glass.

8. The multi-modal system as recited by claim 1, wherein said embedded encapsulated marker is encapsulated by a polymer.

9. The multi-modal system as recited by claim 1, wherein said second system comprises a magnetomechanical marker and is operative at a frequency of about 100 KHz to 125 KHz.

10. The multi-modal system as recited by claim 1, wherein said second system comprises an RFID marker and is operative at a carrier frequency of about 13.56 MHz to 2.456 GHz.

11. The multi-modal system as recited by claim 1, wherein said first system and said second system are operative at dissimilar, mutually distinct frequencies.

12. The multi-modal system as recited by claim 1, wherein said metallic instruments are removed from said surgical wound after being detected by said first system and prior to actuation of said second system.

13. The multi-modal system as recited by claim 1, wherein said first antenna of the first system is affixed to a rollaway cart.

14. The multi-modal system as recited by claim 1, wherein said first antenna of the first system is a hand-held unit.

15. The multi-modal system as recited by claim 1, wherein said second antenna of the second system is affixed to a rollaway cart.

16. The multi-modal system as recited by claim 1, wherein said second antenna of the second system is a hand-held unit.

17. A method of using a multi-modal system, comprising steps of;
a. attaching markers to surgical implements selected for use in a surgical procedure;
b. conducting said surgical procedure;
c. scanning a surgical wound created during said procedure with a first system having a first antenna to detect metallic surgical instruments present within said surgical wound;
d. removing said detected metallic surgical instruments;
e. scanning said surgical wound with a second system having a second antenna to detect non-metallic surgical implements associated with embedded markers present within said surgical wound;
f. removing said detected non-metallic surgical implements;
g. repeating steps “d”, “e” and “f”, immediately prior to closure of said surgical wound; and
h. closing said surgical wound,
whereby retention of surgical metallic instruments and non-metallic surgical implements in a surgical wound cavity following said surgical procedure is avoided.
